(19) United States
(12) Patent Application Publication
(10) Pub. No.: US 2025/0099105 A1
(43) Pub. Date: Mar. 27, 2025

(54) MEDICAL SYSTEM FOR LEFT ATRIAL APPENDAGE OSTIAL CINCHING

(71) Applicant: BOSTON SCIENTIFIC SCIMED, INC., MAPLE GROVE, MN (US)

(72) Inventors: Jerry Wang, Edina, MN (US); James M. Anderson, Corcoran, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 18/889,845

(22) Filed: Sep. 19, 2024

Related U.S. Application Data
(60) Provisional application No. 63/539,706, filed on Sep. 21, 2023.

(51) Int. Cl. A61B 17/12; A61B 17/00

(52) U.S. Cl. CPC A61B 17/12031; A61B 17/12122; A61B 17/12172; A61B 2017/00862

(57) ABSTRACT

A medical system may include a left atrial appendage closure device including an expandable framework configured to shift between a collapsed delivery configuration and an expanded deployed configuration. The expandable framework has an outer diameter in the expanded deployed configuration. The medical system also includes a cinching member configured to shift between a compressed delivery configuration and an expanded configuration. The cinching member is ring shaped with an inner diameter in the expanded configuration that is larger than the outer diameter of the left atrial appendage closure device, and the cinching member has a plurality of tissue engagement elements defined on an outer surface thereof.

### CROSS-REFERENCE TO RELATED APPLICATIONS

This application claims the benefit of priority of U.S. Provisional Application No. 63/539,706 filed Sep. 21, 2023, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices and introducers as well as alternative methods for manufacturing and using medical devices and introducers.

### SUMMARY

This disclosure provides design, material, manufacturing method, and use alternatives for medical devices. An example medical system includes a left atrial appendage closure device including an expandable framework configured to shift between a collapsed delivery configuration and an expanded deployed configuration, the expandable framework having an outer diameter in the expanded deployed configuration, and a cinching member configured to shift between a compressed delivery configuration and an expanded configuration, the cinching member being ring shaped with an inner diameter in the expanded configuration that is larger than the outer diameter of the left atrial appendage closure device, the cinching member have a plurality of tissue engagement elements defined on an outer surface thereof.

Alternatively or additionally to the embodiment above, the cinching member is balloon expandable until an outer diameter of the cinching member is larger than an inner diameter of a patient's left atrial appendage.

Alternatively or additionally to any of the embodiments above, the plurality of tissue engagement elements are radially extending hooks.

Alternatively or additionally to any of the embodiments above, the balloon expandable cinching member is a flexible polymer ring.

Alternatively or additionally to any of the embodiments above, the medical system further includes a plurality of tethers releasably coupled with the balloon expandable cinching member.

Alternatively or additionally to any of the embodiments above, the cinching member is an expandable stent and the plurality of tissue engagement elements are stent capture regions configured to move between a release configuration when the stent is in the collapsed delivery configuration and a pinch configuration when the stent is in the expanded deployed configuration.

Alternatively or additionally to any of the embodiments above, each stent capture region includes adjacent struts that are spaced apart in the release configuration and in close proximity with each other forming a pinching zone in the pinch configuration.

Alternatively or additionally to any of the embodiments above, the adjacent struts extend circumferentially around the stent, and at least some of the stent capture regions include axial struts joining the adjacent struts.

Alternatively or additionally to any of the embodiments above, the stent has a first end and a second end, and at least first and second axial struts extending from the first end to the second end, wherein at least some of the stent capture regions include an irregular shaped enclosed region connected to the first and second axial struts with radial struts, wherein the irregular shaped enclosed region defines an open space when the stent is in the compressed delivery configuration and a compressed space configured to retain tissue when the stent is in the expanded configuration.

Alternatively or additionally to any of the embodiments above, at least some of the stent capture regions include first and second strut extensions connected with a loop, wherein the first and second strut extensions are configured to slide axially relative one another within the loop, each of the first and second struts having a free end with a pinching member.

Alternatively or additionally to any of the embodiments above, the pinching members are hooks.

Alternatively or additionally to any of the embodiments above, the free end of the first strut defines a hoop and the free end of the second strut defines a hook, wherein as the stent moves into the expanded configuration, the first and second strut extensions slide axially relative to one another through the loop, and the hook pinches tissue within the hoop.

Alternatively or additionally to any of the embodiments above, the free end of the first strut includes an aperture and the free end of the second strut includes a projection, wherein when the stent moves into the expanded configuration, the projection sides axially into the aperture.

Alternatively or additionally to any of the embodiments above, the second strut includes an aperture through which the first strut extends, thereby maintaining the first and second struts in a sliding relationship.

Another example medical system includes a left atrial appendage closure device including an expandable framework configured to shift between a collapsed delivery configuration and an expanded deployed configuration, the expandable framework having an outer diameter in the expanded deployed configuration, and a balloon expandable stent configured to shift between a collapsed delivery configuration and an expanded configuration in which an outer diameter of the stent is larger than an inner diameter of a patient's left atrial appendage, the stent having a plurality of stent capture regions configured to move between a release configuration when the stent is in the collapsed delivery configuration and a pinch configuration when the stent is in the expanded deployed configuration.

Alternatively or additionally to any of the embodiments above, each stent capture region includes adjacent struts that are spaced apart in the release configuration and in close proximity with each other forming a pinching zone in the pinch configuration.

Alternatively or additionally to any of the embodiments above, the adjacent struts extend circumferentially around the stent, and at least some of the stent capture regions include axial struts joining the adjacent struts.

Alternatively or additionally to any of the embodiments above, the stent has a first end and a second end, and at least first and second axial struts extending from the first end to the second end, wherein at least some of the stent capture regions include an irregular shaped enclosed region connected to the first and second axial struts with radial struts, wherein the irregular shaped enclosed region defines an open space when the stent is in the compressed delivery configuration and a compressed space configured to retain tissue when the stent is in the expanded configuration.

Alternatively or additionally to any of the embodiments above, at least some of the stent capture regions include first and second strut extensions connected with a loop, wherein the first and second strut extensions are configured to slide axially relative one another within the loop, each of the first and second struts having a free end with a pinching member.

An example method of cinching an ostium of a patient's left atrial appendage around a closure device includes the steps of inserting a cinching member into the ostium of the patient's left atrial appendage, the cinching member being ring shaped and configured to shift between a compressed delivery configuration and an expanded configuration, the cinching member having an outer surface with a plurality of tissue engagement elements, expanding the cinching member thereby engaging the plurality of tissue engagement elements with an inner surface of the patient's left atrial appendage, inserting a closure device into the patient's left atrial appendage within the cinching member, the closure device configured to expand from a collapsed delivery configuration to an expanded deployed configuration, expanding the closure device, and cinching the cinching member thereby pulling the inner surface of the patient's left atrial appendage into contact with an outer surface of the closure device.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic partial cross-sectional view of an example left atrial appendage;

FIG. 2 is a partial cross-sectional view of a left atrial appendage closure device disposed within the left atrial appendage of FIG. 1;

FIG. 3 is a proximal view FIG. 2;

FIG. 4A is a perspective view of an example left atrial appendage closure device;

FIG. 4B is a perspective view of an example cinching member;

FIG. 5A is an end view of the cinching member of FIG. 4B

FIG. 5B is a partial end view of an example tissue engagement element;

FIGS. 6A-6D illustrate a method of inserting a cinching member and closure device into the left atrial appendage and cinching the cinching member around the closure device;

FIG. 7 illustrates inserting a tethered cinching member around a closure device;

FIGS. 8A and 8B are side views of an example stent in the compressed and expanded configurations, respectively;

FIGS. 9A-14B are partial side views of additional example stents in the compressed and expanded configurations; and

FIG. 15 illustrates a system for automatic external cinching of a cinching member around a patient's left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the current disclosure. Those skilled in the art will recognize that the various elements described and/or shown may be arranged in various combinations and configurations without departing from the scope of the disclosure. The detailed description and drawings illustrate example embodiments of the current disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed example(s). Each instance of the features may include and/or be encompassed by the singular disclosure(s), unless expressly stated to the contrary. For simplicity and clarity purposes, not all elements of the current disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

When implanting closure devices in the left atrial appendage (LAA), complete sealing is desired. Tissue manipulation approaches to sealing the LAA without inserting an implant, such as cinching the neck of the LAA with a suture, may be overly damaging to tissue due to the high tissue forces created. Further, such tissue manipulation may remodel the heart in unexpected ways or may create tissue necrosis. When inserting an implant into the LAA, some anatomies do not achieve a complete seal due to eccentric ostiums, tissue motion, remodeling, etc. Many LAA sealing devices have a substantially circular outer geometry, while some patients may have an LAA ostium with a more oval geometry, often with an uneven border. While tissue ingrowth into the sealing device may create a seal around most of the ostium, blood may still leak around the device and into the LAA.

The following figures illustrate selected components and/or arrangements of a medical system for providing complete occlusion of the left atrial appendage, and/or methods of using the medical system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

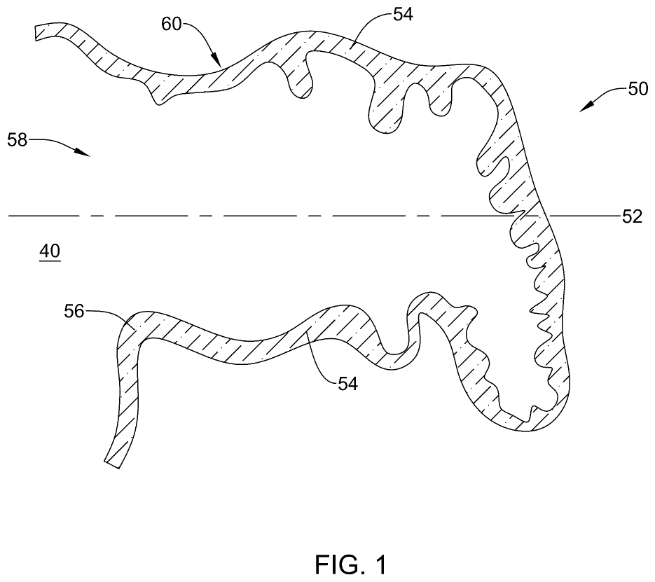

Turning now to the figures, FIG. 1 is a partial cross-sectional view of an example left atrial appendage (LAA) 50, which may be attached to and in fluid communication with the left atrium 40 of a patient's heart. The left atrial appendage 50 may have a complex geometry and/or irregular surface area. Those skilled in the art will recognize that the illustrated LAA is merely one of many possible shapes and sizes for the LAA, which may vary from patient to patient. Those of skill in the art will also recognize that the medical devices and methods disclosed herein may be adapted for various sizes and shapes of the LAA, as necessary. A left atrial appendage 50 may include a generally longitudinal axis 52 arranged along a depth of a main body 60 of the left atrial appendage 50. The main body 60 may include a lateral wall 54 and an ostium 56 forming a proximal mouth 58. In some embodiments, a lateral extent of the ostium 56 and/or the lateral wall 54 may be smaller or less than a depth of the main body 60 along the longitudinal axis 52, or a depth of the main body 60 may be greater than a lateral extent of the ostium 56 and/or the lateral wall 54. In some embodiments, the left atrial appendage 50 may narrow quickly along the depth of the main body 60 or the left atrial appendage may maintain a generally constant lateral extent along a majority of depth of the main body 60. In some embodiments, the left atrial appendage 50 may include a distalmost region formed or arranged as a tail-like clement associated with a distal portion of the main body 60. In some embodiments, the distalmost region may protrude radially or laterally away from the longitudinal axis 52.

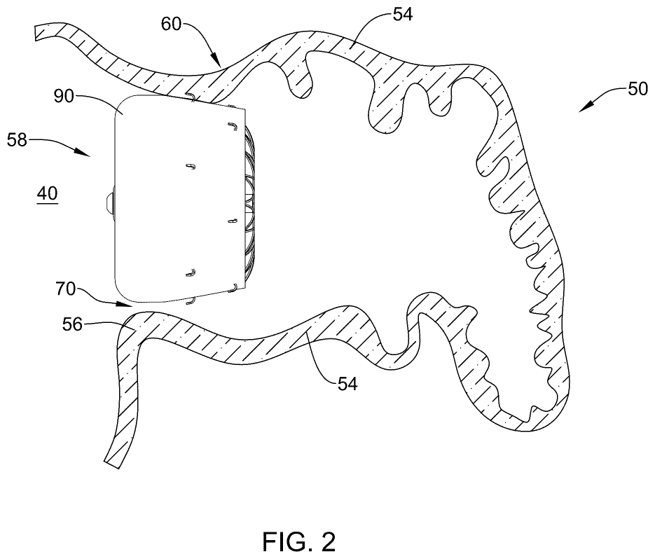

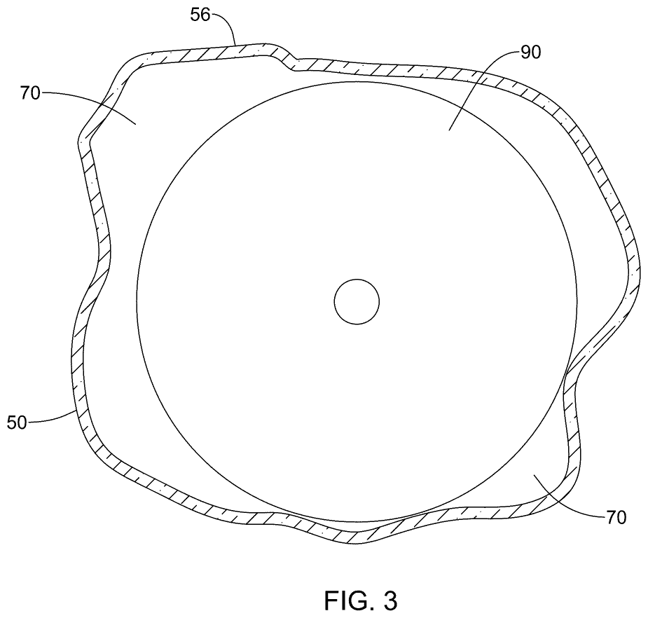

As shown in FIG. 2, an LAA closure device 90 may be implanted within the ostium 56 of the LAA 50 to close off the proximal mouth 58. Ideally the closure device 90 would completely seal off the left atrial appendage 50 from the left atrium 40 and/or the patient's circulatory system. However, due to the patient's anatomy and/or placement of the closure device 90, complete closure may not be achieved, with one or more gaps 70 between the outer edge of the closure device 90 and the lateral wall 54, as shown in FIG. 2. FIG. 3 illustrates a proximal view of FIG. 2, showing the closure device 90 disposed within the ostium 56 of the LAA 50, showing gaps 70 between the outer edge of the closure device 90 and the ostium 56.

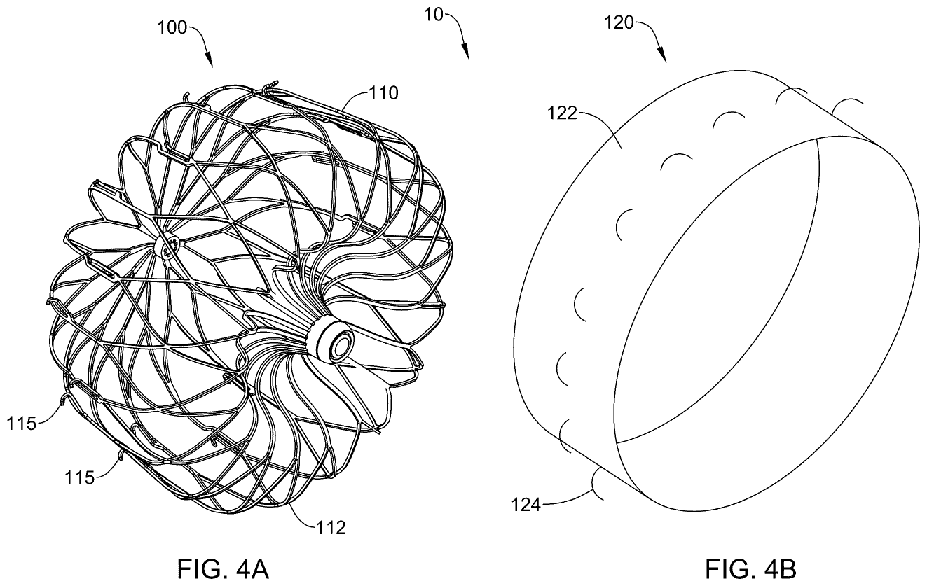

FIGS. 4A and 4B illustrate the components of a medical system 10 that may achieve complete closure of a patient's LAA, without leaks. The medical system may include an expandable LAA closure device 100 and a cinching member 120. The LAA closure device 100 may include an expandable framework 110 configured to shift between a collapsed delivery configuration and an expanded deployed configuration. The expandable framework 110, when in the expanded deployed configuration, may be configured to fill a majority of an inner diameter of a patient's LAA.

The LAA closure device 100 may be self-expanding as shown in FIG. 4A, or it may be balloon expandable. The LAA closure device 100 may include a plurality of tissue engagement members 115 extending radially outward from the outer surface of the expandable framework 110. The tissue engagement members 115 may be configured to engage the inner surface of the LAA, such as the hooks 115 shown in FIG. 4A. the LAA closure device 100 may include a covering (not shown) disposed over at least the proximal end face 112. The expandable framework 110 may be compliant and may be configured to substantially conform to and/or be in sealing engagement with the shape and/or geometry of the lateral wall 54 and/or the ostium 56 of the LAA when the expandable framework 110 is in the expanded deployed configuration. In some embodiments, the LAA closure device 100 may expand to a size, extent, or shape less than or different from a maximum unconstrained extent, as determined by the surrounding tissue and/or lateral wall of the left atrial appendage.

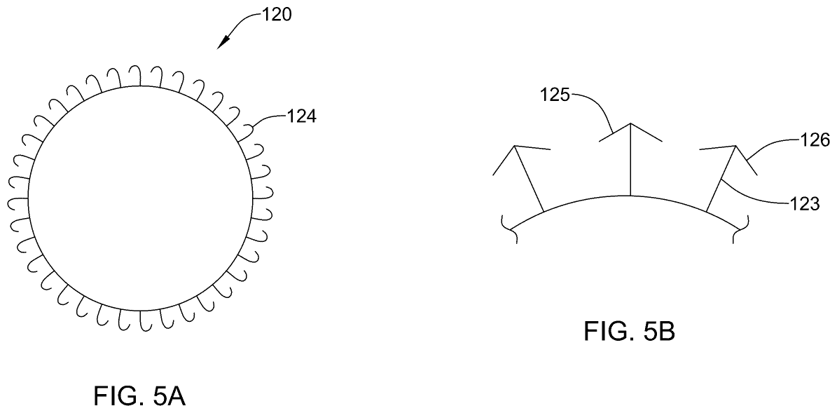

The cinching member 120 may be configured to shift between a radially compressed delivery configuration and an expanded deployed configuration. In some embodiments, the cinching member 120 may be an expandable ring-shaped band 122 with an inner diameter in the expanded configuration that is larger than the outer diameter of the LAA closure device 100. The cinching member 120 have a plurality of tissue engagement elements 124 defined on an outer surface thereof. In some embodiments, the cinching member 120 may be balloon expandable. Using a balloon to expand the cinching member 120 may allow for expansion until an outer diameter of the cinching member 120 is larger than an inner diameter of the patient's LAA. Slight expansion or stretching of the LAA may ensure the cinching member 120 engages tissue around the entire ostium. In some embodiments, the plurality of tissue engagement elements 124 may be radially extending hooks, as illustrated in FIG. 4B. FIG. 5A illustrates an embodiment of the cinching member 120 in which all of the hooks 124 are oriented in the same direction. This may allow for the cinching member 120 to be rotated to embed the hooks 124 into the tissue of the LAA. The plurality of hooks 124 may also be oriented in a variety of directions (not shown). FIG. 5B illustrates a portion of the cinching member 120 in which the tissue engagement elements are barbs 125. The barbs 125 may each have a radially extending shaft 123 with an arrowhead 126 fixed to the shaft 123.

The cinching member 120 may be a balloon expandable flexible polymer ring configured to engage lobes and folds or valleys of the inner surface of the LAA. In other embodiments, the cinching member 120 may be made of a shape memory material, including metals and polymers. In other embodiments, the cinching member 120 may be a woven mesh or a fabric sleeve. In all embodiments, the outer surface of the cinching member 120 may have affixed thereto the plurality of tissue engagement elements 124, such that balloon expansion of the cinching member 120 pushes the tissue engagement elements 124 into the inner surface of the LAA, thereby securing the cinching member 120 to the LAA.

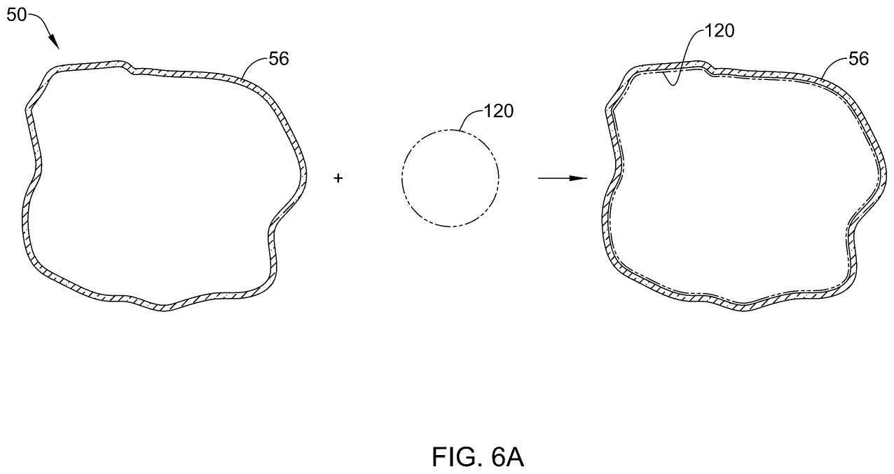

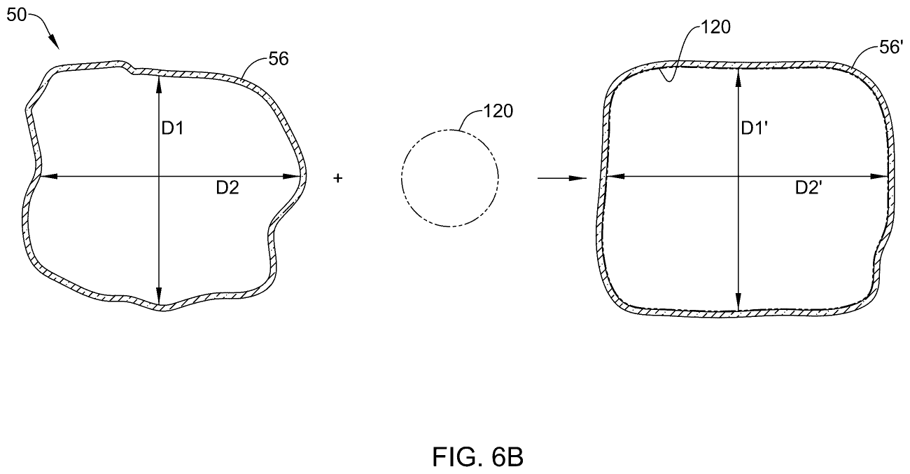

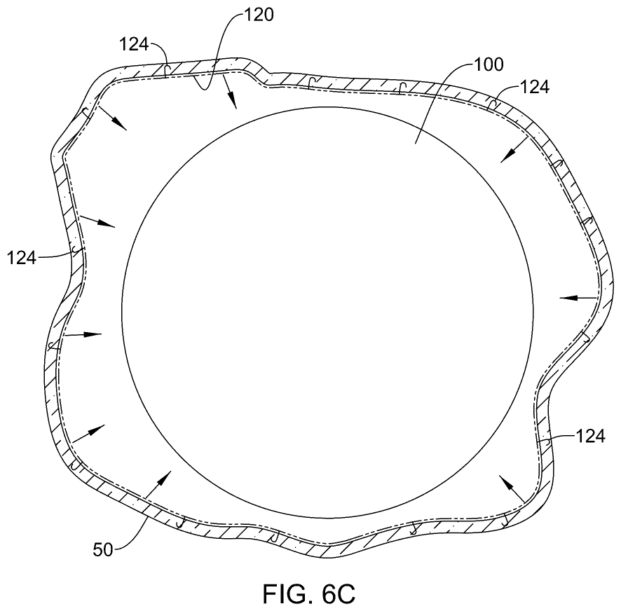

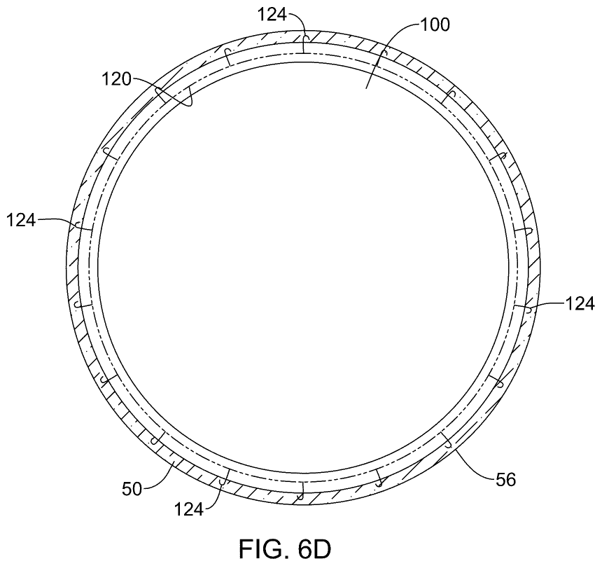

FIGS. 6A-6D illustrate a method of inserting the cinching member 120 and the LAA closure device 100 into the ostium 56 of the LAA 50. In FIG. 6A, the cinching member 120 is inserted into and expanded against the inner surface of the ostium 56 with the tissue engagement elements securing the cinching member 120 to the ostium. FIG. 6B illustrates the embodiment described above in which the cinching member 120 is expanded to a diameter D1′, D2′ greater than the inner diameter D1, D2 of the LAA ostium 56. As shown in FIG. 6B the expanded ostium 56′ has diameters D1′ and D2′ that are larger than the original diameters D1 and D2 in the LAA prior to insertion and expansion of the cinching member 120. Once the cinching member 120 has been expanded and is engaged with the inner surface of the LAA, the LAA closure device 100 may be inserted into the LAA 50, inside the cinching member 120, as shown in FIG. 6C. The final step in the method is to cinch or compress the cinching member 120 against the LAA closure device 100 which pulls the ostium 56 of the LAA 50 tight against the closure device 100 such that there are no leaks between the outer surface of the closure device 100 and the inner surface of the LAA, as shown in FIG. 6D.

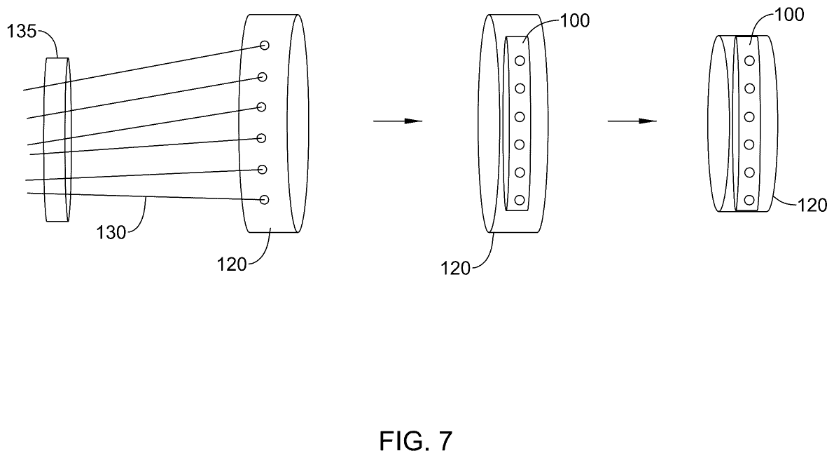

In some embodiments, medical system may include a plurality of tethers 130 releasably coupled with the cinching member 120, as shown in FIG. 7. The tethers 130 may be coupled to a handle 135. The tethers 130 may be releasably coupled to the inner surface of the cinching member 120, such as with a threaded connection or a frangible connection that is broken upon the application of a preset amount of force. The tethers 130 remain coupled to the cinching member 120 until the cinching member 120 has been cinched or pulled into contact with the LAA closure device 100, at which time the tethers 130 may be released and withdrawn from the body, leaving the cinching member 120 and the LAA closure device 100 in place sealing off the LAA.

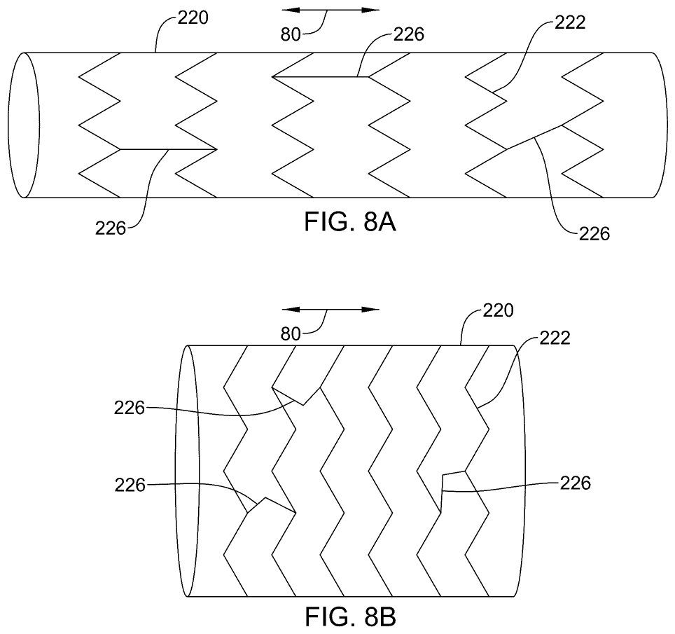

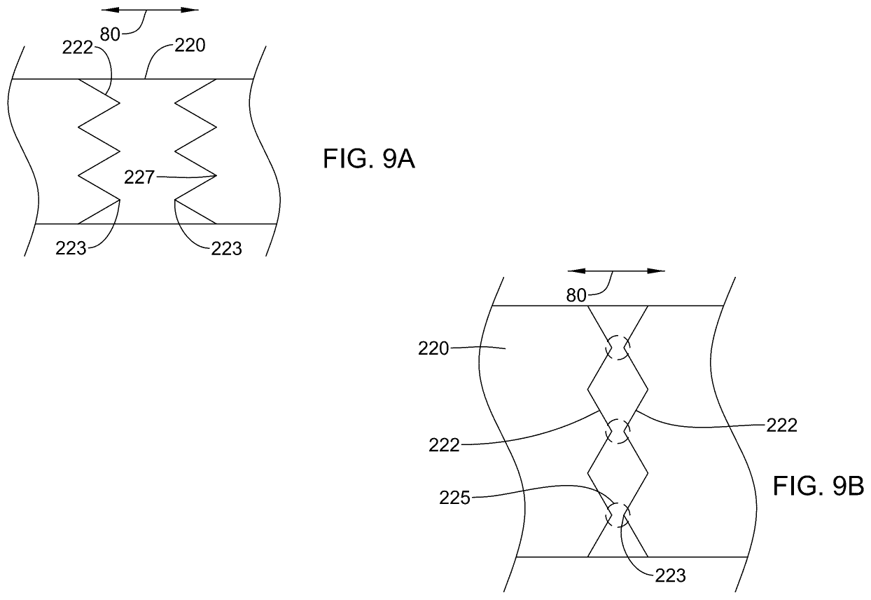

FIGS. 8A-15 illustrate various embodiments in which the cinching member is an expandable stent and the plurality of tissue engagement elements are stent capture regions configured to move between a release configuration when the stent is in the radially collapsed delivery configuration and a pinch configuration when the stent is in the radially expanded deployed configuration. In each embodiment, the stent moves between an axially extended, radially collapsed configuration for delivery, to an axially compressed, radially expanded deployed configuration. The axial movement is indicated by arrow 80 in each figure. FIG. 8A shows an example stent 220 in the radially collapsed delivery configuration with a plurality of struts 222 in a zig-zag pattern. FIG. 8B shows the stent 220 of FIG. 8A in the radially expanded deployed configuration. The struts 222 may extend helically between ends of the stent 220 or they may extend circumferentially around the stent and have one or more longitudinal struts 226 connecting adjacent struts 222, where the longitudinal struts 226 may have bend regions or other compression structures to accommodate axial compression and radial expansion of the stent 220, as indicated by arrow 80. This helical or circumferential pattern of struts 222, with or without one or more longitudinal struts 226 may apply to any of the stents described in FIGS. 9A-14B. FIGS. 9A and 9B show a portion of the stent 220 with one embodiment of a strut pattern defining a plurality of stent capture regions 225. FIG. 9A shows the stent 220 in the collapsed delivery configuration with the plurality of struts 222 in the compressed zig-zag pattern, with circumferentially extending struts 222 spaced axially apart. FIGZ. 9B shows the stent 220 in the expanded deployed configuration where the radial expansion has caused foreshortening of the stent such that the zig-zag pattern of the struts 222 is radially expanded. Adjacent struts have moved towards one another to form stent capture regions 225 where points of adjacent circumferential struts are in close proximity with one another and will capture or pinch tissue therebetween as the stent expands and foreshortens.

As shown in FIG. 9A, the stent struts 222 define a series of alternating peaks 223 and valleys 227. When the stent moves from the radially collapsed delivery configuration (FIG. 9A) to the radially expanded deployed configuration (FIG. 9B), the stent undergoes axial foreshortening and the longitudinally adjacent peaks 223 move axially towards one another and create the stent capture regions 225 between longitudinally adjacent peaks 223. Each stent capture region 225 thus moves from the release configuration in which adjacent struts 222 are spaced apart, to the pinch configuration in which the adjacent struts 222 are in close proximity with each other. The stent capture regions 225 define pinching zones where tissue of the LAA is pinched between adjacent stent peaks 223 in the pinch configuration. In the embodiment shown in FIGS. 9A and 9B, the stent capture regions 225 are between adjacent peaks 223.

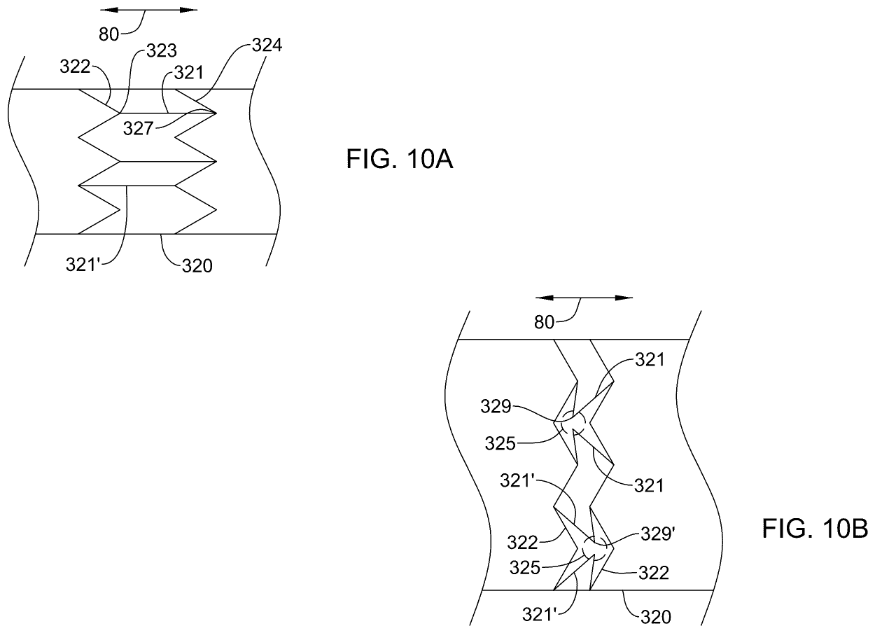

FIGS. 10A and 10B show an embodiment of stent 320 in which the stent capture regions 325 are formed by axial struts 321 that extend between the 323 on one strut 322 and the valley 327 on an axially adjacent strut 322. In this embodiment, axial struts 321 are configured to bend as the stent 320 expands into the deployed configuration, forming a protrusion 329, as shown in FIG. 10B. Circumferentially adjacent axial struts 321 may be formed with preset bends such that in the expanded deployed configuration, the protrusions 329 move into close proximity to form the stent capture regions 325 where tissue is pinched as the stent expands during deployment. The axial struts 321 may extend between adjacent peaks 323 and valleys 327, between adjacent peaks, between adjacent valleys, or any combination thereof. In the embodiment shown in FIGS. 10A and 10B, the stent 320 includes axial struts 321 between the peak 323 on a first strut 322 and the corresponding valley 327 on the adjacent second strut 324, as well as axial struts 321′ extending between the valley 327 on the first strut 322 and the corresponding peak 323 on the second strut 324. This arrangement provides protrusions 329, 329′ extending in opposite directions, which may aid in securing the stent 320 to the tissue of the LAA.

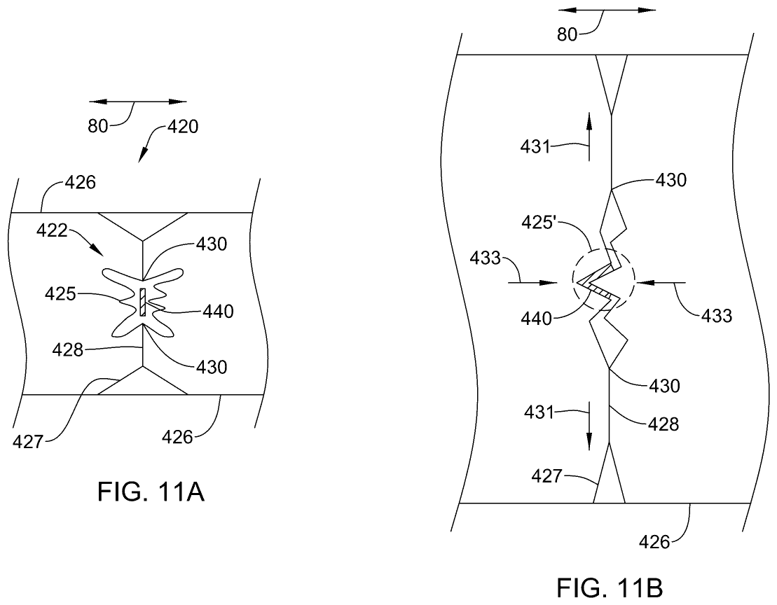

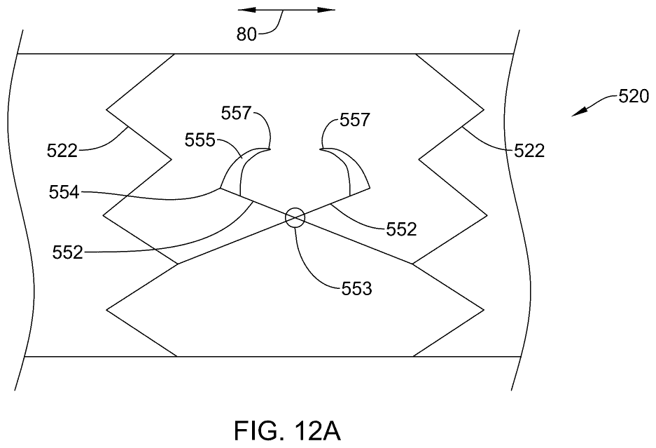

FIGS. 11A and 11B show a portion of a stent 420 with a different strut configuration that forms a plurality of stent capture regions 425 as the stent is expanded from the compressed delivery configuration (FIG. 11A) to the expanded deployed configuration (FIG. 11B). This stent 420 has a first end and a second end, and at least first and second longitudinal struts 426 extending from the first end to the second end, and a plurality of radial struts 422 connecting the first and second longitudinal struts 426. The radial struts 422 may each define at least one stent irregular shaped enclosed region 425 that transitions into a stent capture region 425′. The irregular shaped enclosed region 425 defines an open space configured to enclose tissue 440 when the stent is in the compressed delivery configuration, as shown in FIG. 11A. The irregular shaped enclosed region 425 may be connected to the longitudinal struts 426 with first and second circumferential regions 428 and angled regions 429. As the stent expands radially, the first and second circumferential regions 428 pull opposing ends 430 of the irregular shaped enclosed region 425 radially, as indicated by arrows 431. The radial movement of the opposing ends 430 causes axial compression of the irregular shaped enclosed region 425, as indicated by arrows 433. The irregular shaped enclosed region 425′ is configured to pinch and retain tissue 440 within the open space when the stent is in the expanded configuration, as shown in FIG. 11B.

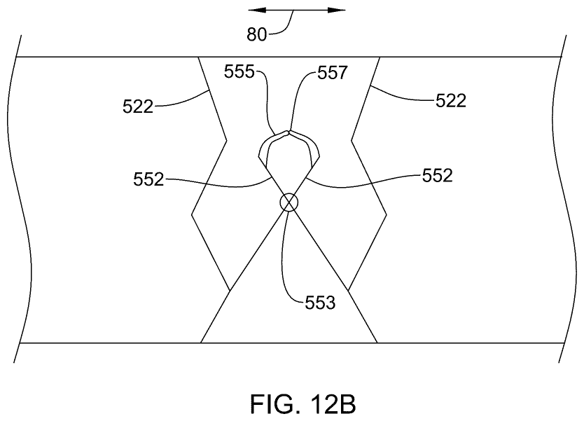

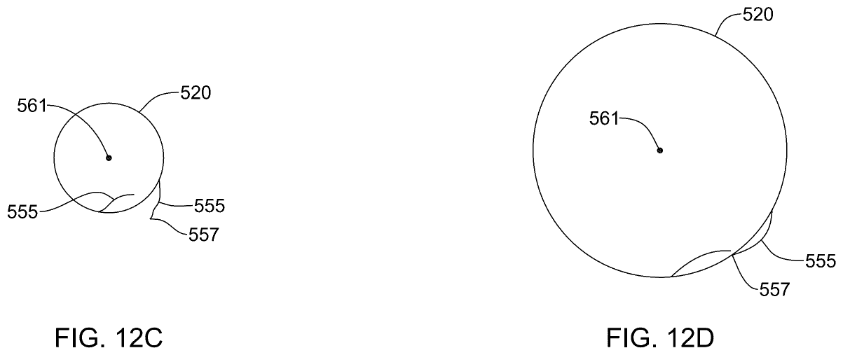

FIGS. 12A and 12B illustrate a portion of a stent 520 with a sliding pincher structure for pinching and securing the stent to tissue of the LAA. The stent includes a plurality of circumferential struts 522 where first and second adjacent struts have first and second strut extensions 552, respectively, extending into the space between the adjacent struts 522. The first and second strut extensions 552 cross one another and extend through a ring 553 such that the strut extensions 552 may slide axially relative one another within the ring 553 as the stent expands radially and the circumferential struts 522 move toward one another. Each of the strut extensions 552 has one end fixed to a strut 522 and a free end 554 defining a pinching member 555. In the embodiment shown in FIGS. 12A and 12B, the pinching members 555 are hooks with tips 557 that may be sharpened or blunt. In the compressed delivery configuration (FIG. 12A), the tips 557 of the pinching members 555 are spaced apart axially. As the stent expands radially into the expanded deployed configuration (FIG. 12B), the first and second struts 522 move toward each other, causing the first and second strut extensions 552 to slide relative to one another through the ring 553 until the tips 557 of the pinching members 555 are in close proximity to one another or touch one another. When the stent is inserted into and expanded within the LAA, the tips 557 pinch tissue therebetween as the stent moves into the expanded configuration. The strut extensions 552, pinching members 555, and tips 557 may move relative to one another in a single plane, sliding axially relative to one another.

In another embodiment, at least the pinching members 555 and tips 557 may move transversely towards or away from a central longitudinal axis 561 of the stent 520 as the first and second stent extensions 552 slide through the ring 553. For example, the stent extensions 552 or just the pinching members 555 may be configured to rotate about their longitudinal axis to move the tips 557 away from and toward each other. Rotation may be provided by the join between the stent extension 552 and the strut 522. Expansion of the struts 522 may twist the stent extensions 552, thereby rotating the tips 557 towards one another. In another example, the stent extensions 552 may include a bend adjacent the pinching members 555 such that when the stent 520 is in the compressed delivery configuration, at least the tips 557 are separated from one another in a direction transverse to the longitudinal axis 561, as shown in FIG. 12C. As the struts 522 expand and the stent 520 increases in diameter and decreases in length, the tips 557 may rotate towards one another to pinch tissue therebetween, as shown in FIG. 12D.

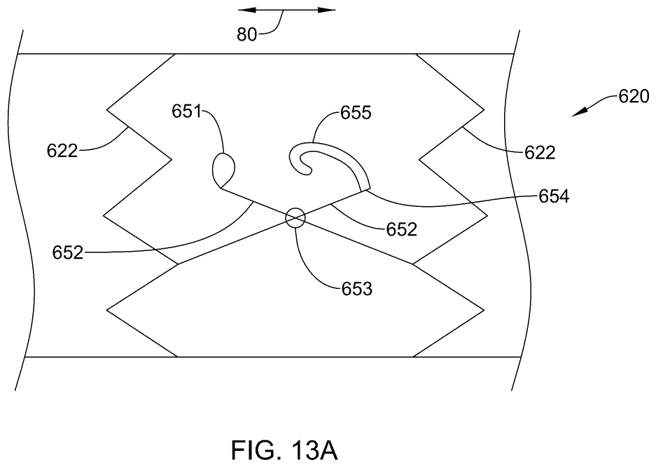

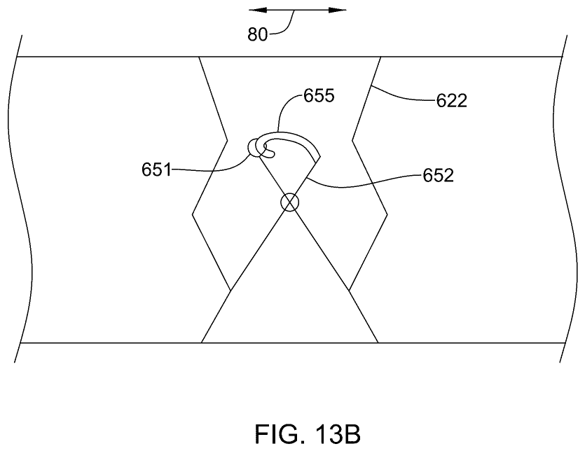

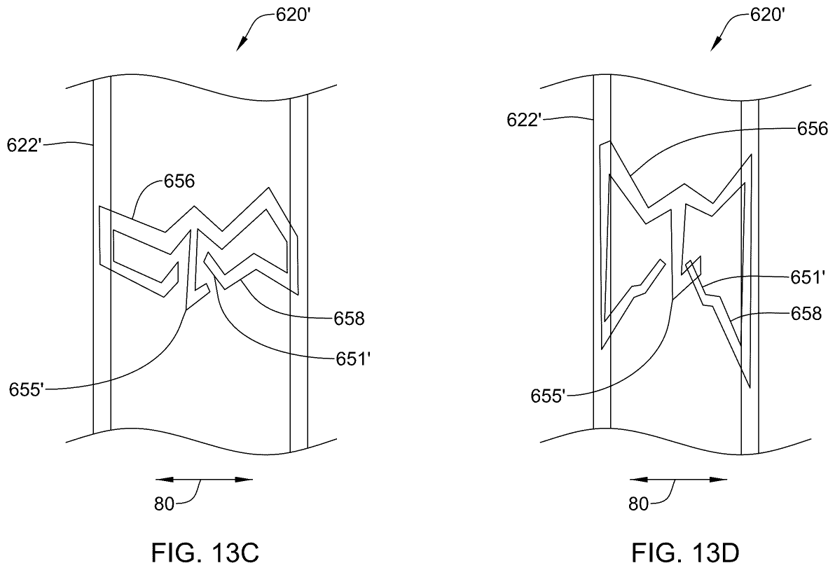

FIGS. 13A and 13B illustrate a portion of another stent 620 with a sliding pincher structure for pinching and securing the stent to tissue of the LAA. The stent includes a plurality of circumferential struts 622 where first and second adjacent struts have first and second strut extensions 652, respectively, extending into the space between the adjacent struts 622. The first and second strut extensions 652 cross one another and extend through a ring 653 such that the strut extensions 652 may slide axially relative one another within the ring 653 as the stent expands radially and the circumferential struts 522 move toward one another. Each of the strut extensions 652 has one end fixed to a strut 622 and a free end 654. One of the strut extensions 652 has a hook 655 on its free end and the other strut extension 652 has a loop 651 on its free end. In the compressed delivery configuration (FIG. 13A), the hook 655 and loop 651 are spaced apart axially. As the stent moves into the expanded deployed configuration (FIG. 13B), the first and second struts 622 move toward each other, causing the first and second strut extensions 652 to slide axially relative to one another through the ring 653, and the hook 655 pinches tissue within the loop 651.

FIGS. 13C and 13D illustrate a portion of another stent 620′ with a variation on the sliding pincher structure for pinching and securing the stent to tissue of the LAA. This stent 620′ includes a plurality of circumferential struts 622′ where first and second adjacent struts have first and second strut extensions 656, 658, respectively, extending into the space between the adjacent struts 622′. The first strut extension 656 may include a lateral extension defining a hook 655′ on its free end and the second strut extension 658 may include a loop 651′ at its free end. In the compressed delivery configuration (FIG. 13C), the hook 655′ and loop 651′ are spaced apart. As the stent moves into the expanded deployed configuration (FIG. 13D), the first and second struts 622′ move toward each other, causing the hook 655′ to move into the loop 651′, pinching tissue therebetween and within the loop 651′.

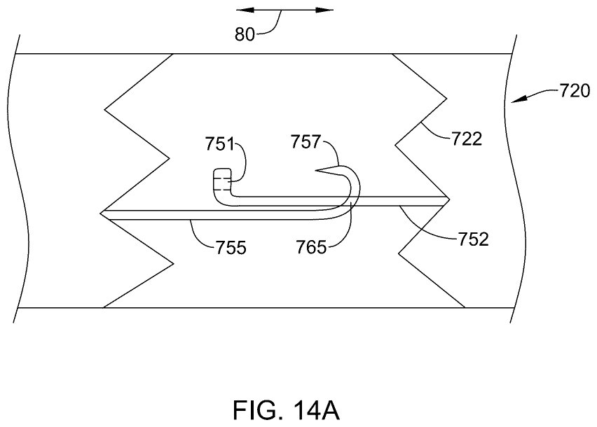

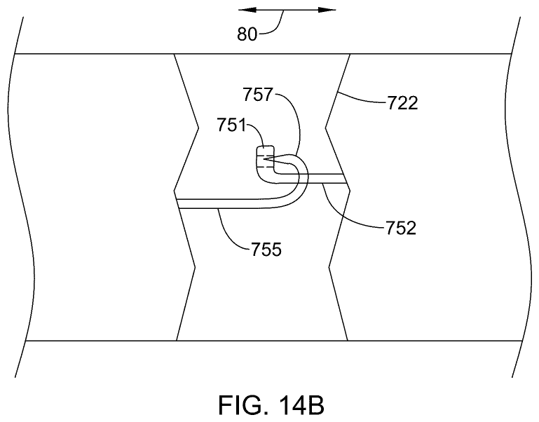

FIGS. 14A and 14B illustrate a portion of an additional embodiment of a stent 720 with a sliding pincher structure for pinching and securing the stent to tissue of the LAA. The stent includes a plurality of circumferential struts 722 where first and second adjacent struts have a first strut extension 752 and a second strut extension 755, respectively, extending axially into the space between the adjacent struts 722. The first strut extension 752 may slide through an opening 765 in the second stent extension 755 such that the first and second strut extensions 752 may slide axially relative one another as the stent expands radially and the circumferential struts 722 move toward one another. The first strut extension 752 has one end fixed to a strut 722 and a free end defining an aperture 751. The second strut extension 755 has one end fixed to a strut 722 and a free end defining a projection 757 configured to fit inside the aperture 751. The projection 757 may have a sharpened or blunt tip. In the compressed delivery configuration (FIG. 14A), the projection 757 is spaced apart axially from the aperture 751. As the stent expands radially into the expanded deployed configuration (FIG. 14B), the first and second struts 722 move toward each other, causing the first and second strut extensions 752, 755 to slide relative to one another until the projection 757 of the second stent extension 755 enters the aperture 751 on the first strut extension 752. When the stent is inserted into and expanded within the LAA, tissue may be pinched between the free ends of the first and second stent extensions 752, 755, and the projection 757 may engage tissue as it moves into the aperture 751.

In some embodiments, the struts of any of the above described stents may be cut to be an exaggerated trapezoid with a parallelogram cross-section to enhance twisting out of plane during expansion. This twisting motion may increase engagement of the struts with the inner surface of the LAA during stent expansion.

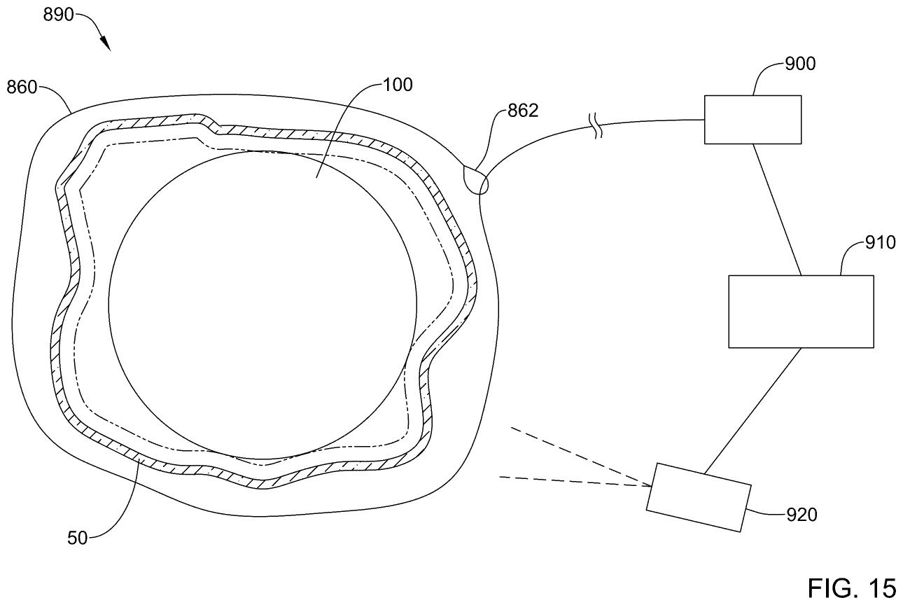

FIG. 15 illustrates an automatic cinching system 890 that involves placing a cinching member 860 around the outside of the LAA 50 with a closure member 100 positioned within the LAA. The cinching member 860 may have a loop 862 disposed at a free end thereof and the second end may be threaded through the loop 862 and connected to an actuator 900 positioned outside the body. The actuator 900 may be coupled to a controller 910 configured to actuate the actuator 900 to apply tension to the cinching member 860, cinching the cinching member 860 around the LAA 50 thereby closing any gaps between the outer surface of the closure member 100 and the inner surface of the LAA. As the actuator is cinching the cinch member 860 around the LAA, color dopppler 920 may be used to determine when any leaks have stopped, at which time the controller 910 may automatically stop the actuator 900, and the cinching member 860 may be secured and tied off.

The materials that can be used for the various components of the medical system 10 and the various elements thereof disclosed herein may include those commonly associated with medical devices. For simplicity purposes, the following discussion refers to the medical system. However, this is not intended to limit the devices and methods described herein, as the discussion may be applied to other elements, members, components, or devices disclosed herein, such as, but not limited to, the cinching member 120, stent (220, 320, 420, 520, 620, 720) the left atrial appendage closure device 100, and/or elements or components thereof.

In some embodiments, the medical system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chronium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical system and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical system to achieve the same result.

In some embodiments, a degree of Magnetic Resonance Imaging (MRI) compatibility is imparted into the medical system and/or other elements disclosed herein. For example, the medical system and/or components or portions thereof may be made of a material that does not substantially distort the image and create substantial artifacts (i.e., gaps in the image). Certain ferromagnetic materials, for example, may not be suitable because they may create artifacts in an MRI image. The medical system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the medical system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Co-Cr or Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the medical system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical system, comprising:
a left atrial appendage closure device including an expandable framework configured to shift between a collapsed delivery configuration and an expanded deployed configuration, the expandable framework having an outer diameter in the expanded deployed configuration; and
a cinching member configured to shift between a compressed delivery configuration and an expanded configuration, the cinching member being ring shaped with an inner diameter in the expanded configuration that is larger than the outer diameter of the left atrial appendage closure device, the cinching member have a plurality of tissue engagement elements defined on an outer surface thereof.

2. The medical system of claim 1, wherein the cinching member is balloon expandable until an outer diameter of the cinching member is larger than an inner diameter of a patient's left atrial appendage.

3. The medical system of claim 1, wherein the plurality of tissue engagement elements are radially extending hooks.

4. The medical system of claim 2, wherein the balloon expandable cinching member is a flexible polymer ring.

5. The medical system of claim 4, further comprising a plurality of tethers releasably coupled with the balloon expandable cinching member.

6. The medical system of claim 1, wherein the cinching member is an expandable stent and the plurality of tissue engagement elements are stent capture regions configured to move between a release configuration when the stent is in the collapsed delivery configuration and a pinch configuration when the stent is in the expanded deployed configuration.

7. The medical system of claim 6, wherein each stent capture region includes adjacent struts that are spaced apart in the release configuration and in close proximity with each other forming a pinching zone in the pinch configuration.

8. The medical system of claim 7, wherein the adjacent struts extend circumferentially around the stent, and at least some of the stent capture regions include axial struts joining the adjacent struts.

9. The medical system of claim 6, wherein the stent has a first end and a second end, and at least first and second axial struts extending from the first end to the second end, wherein at least some of the stent capture regions include an irregular shaped enclosed region connected to the first and second axial struts with radial struts, wherein the irregular shaped enclosed region defines an open space when the stent is in the compressed delivery configuration and a compressed space configured to retain tissue when the stent is in the expanded configuration.

10. The medical system of claim 6, wherein at least some of the stent capture regions include first and second strut extensions connected with a loop, wherein the first and second strut extensions are configured to slide axially relative one another within the loop, each of the first and second struts having a free end with a pinching member.

11. The medical system of claim 10, wherein the pinching members are hooks.

12. The medical system of claim 10, wherein the free end of the first strut defines a hoop and the free end of the second strut defines a hook, wherein as the stent moves into the expanded configuration, the first and second strut extensions slide axially relative to one another through the loop, and the hook pinches tissue within the hoop.

13. The medical system of claim 10, wherein the free end of the first strut includes an aperture and the free end of the second strut includes a projection, wherein when the stent moves into the expanded configuration, the projection sides axially into the aperture.

14. The medical system of claim 13, wherein the second strut includes an aperture through which the first strut extends, thereby maintaining the first and second struts in a sliding relationship.

15. A medical system, comprising:
a left atrial appendage closure device including an expandable framework configured to shift between a collapsed delivery configuration and an expanded deployed configuration, the expandable framework having an outer diameter in the expanded deployed configuration; and
a balloon expandable stent configured to shift between a collapsed delivery configuration and an expanded configuration in which an outer diameter of the stent is larger than an inner diameter of a patient's left atrial appendage, the stent having a plurality of stent capture regions configured to move between a release configuration when the stent is in the collapsed delivery configuration and a pinch configuration when the stent is in the expanded deployed configuration.

16. The medical system of claim 15, wherein each stent capture region includes adjacent struts that are spaced apart in the release configuration and in close proximity with each other forming a pinching zone in the pinch configuration.

17. The medical system of claim 16, wherein the adjacent struts extend circumferentially around the stent, and at least some of the stent capture regions include axial struts joining the adjacent struts.

18. The medical system of claim 15, wherein the stent has a first end and a second end, and at least first and second axial struts extending from the first end to the second end, wherein at least some of the stent capture regions include an irregular shaped enclosed region connected to the first and second axial struts with radial struts, wherein the irregular shaped enclosed region defines an open space when the stent is in the compressed delivery configuration and a compressed space configured to retain tissue when the stent is in the expanded configuration.

19. The medical system of claim 15, wherein at least some of the stent capture regions include first and second strut extensions connected with a loop, wherein the first and second strut extensions are configured to slide axially relative one another within the loop, each of the first and second struts having a free end with a pinching member.

20. A method of cinching an ostium of a patient's left atrial appendage around a closure device, comprising:
inserting a cinching member into the ostium of the patient's left atrial appendage, the cinching member being ring shaped and configured to shift between a compressed delivery configuration and an expanded configuration, the cinching member having an outer surface with a plurality of tissue engagement elements;
expanding the cinching member thereby engaging the plurality of tissue engagement elements with an inner surface of the patient's left atrial appendage;
inserting a closure device into the patient's left atrial appendage within the cinching member, the closure device configured to expand from a collapsed delivery configuration to an expanded deployed configuration;
expanding the closure device; and
cinching the cinching member thereby pulling the inner surface of the patient's left atrial appendage into contact with an outer surface of the closure device.
